(19) United States
(12) Patent Application Publication
(10) Pub. No.: US 2012/0130231 A1
(43) Pub. Date: May 24, 2012

(54) MAGNETIC NAVIGATION ENABLED DELIVERY TOOLS AND METHODS OF MAKING AND USING SUCH TOOLS

(75) Inventors: Thao Ngo, Shakopee, MN (US); Tyler Strang, Valencia, CA (US); Vitaliy Epshteyn, Maple Grove, CA (US); Lior Sobe, Kadima (IL); Ran Sela, Tel-Aviv (IL); Guy Vanney, Blaine, MN (US)

(73) Assignee: PACESETTER, INC., Sylmar, CA (US)

(21) Appl. No.: 12/953,943

(22) Filed: Nov. 24, 2010

(51) Int. Cl. A61B 17/00; A61B 5/055; H01R 43/00

(52) U.S. Cl. 600/424; 606/129; 29/825

(57) ABSTRACT

Disclosed herein is a magnetic navigation enabled tool configured for the delivery of an implantable medical lead. The tool includes a tubular body, a sensor and a conductor. The tubular body includes a distal end, a proximal end, an inner layer including an outer circumferential surface, a lumen inward of the inner layer, and an outer layer over the outer circumferential surface of the inner layer. The sensor is on the tubular body near the distal end. The conductor extends from the sensor coil towards the proximal end imbedded in the inner layer.

### FIELD OF THE INVENTION

The present invention relates to medical apparatus and methods. More specifically, the present invention relates to delivery tools for the implantation of medical leads and methods of manufacturing and using such delivery tools.

### BACKGROUND OF THE INVENTION

Implantable pulse generators, such as pacemakers, implantable cardioverter defibrillators (“ICD”) and neurostimulators, provide electrotherapy via implantable medical leads to nerves, such as those nerves found in cardiac tissue, the spinal column, the brain, etc. Electrotherapy is provided in the form of electrical signals, which are generated in the pulse generator and travel via the medical lead's conductors to the electrotherapy treatment site.

In the realm of cardiology, medical leads are implanted in the heart via delivery tools, such as, for example, catheters, sheaths, guidewires, and stylets. A guidewire is typically negotiated through the vasculature and cardiac structure of the patient to the implantation location within the heart of the patient. The medical lead is then tracked over the guidewire with the pushing assistance of a stylet. This process of delivering the medical lead to the implantation site is visualized via two dimensional (“2D”) X-ray fluoroscopy, which exposes the patient to toxic dye and the patient and attending medical staff to continuous radiation. The 2D fluoroscopic images leave much to be desired with respect to communicating to the physician the information needed to negotiate the delivery tools and medical lead to the implantation site. As a result, the time necessary for a lead implantation procedure from patient to patient can be unpredictable.

There is a need in the art for systems, tools and methods that reduce the exposure to toxic dye and radiation. There is also a need in the art for systems, tools and methods that facilitate improved communication to the physician of the information needed to navigate or negotiate the delivery tools and medical lead to the implantation site. There is also a need in the art for methods of manufacturing such systems and tools.

### BRIEF SUMMARY OF THE INVENTION

Disclosed herein is a magnetic navigation enabled tool configured for the delivery of an implantable medical lead. In one embodiment, the tool includes a tubular body, a sensor and a conductor. The tubular body includes a distal end, a proximal end, an inner layer including an outer circumferential surface, a lumen inward of the inner layer, and an outer layer over the outer circumferential surface of the inner layer. The sensor is on the tubular body near the distal end. The conductor extends from the sensor coil towards the proximal end imbedded in the inner layer.

Also disclosed herein is a magnetic navigation enabled tool configured for the delivery of an implantable medical lead. In one embodiment, the tool includes a hypotube, a sensor, a conductor and a fill material. The hypotube includes a recess defined in a wall of the hypotube and extending longitudinally along the hypotube. The sensor is near a distal end of the hypotube. A conductor is routed along the recess from the sensor towards a proximal end of the hypotube. The fill material imbeds the conductor in the recess and generally fills the recess.

Further disclosed herein is a magnetic navigation enabled tool configured for the delivery of an implantable medical lead. In one embodiment, the tool includes a hypotube, a sensor, a conductor and a material forming an outer layer of the tool. The hypotube includes a lumen and an outer circumferential surface. The sensor is near a distal end of the hypotube. The conductor is routed along the outer circumferential surface from the sensor towards a proximal end of the hypotube. The material extends over the conductor and outer circumferential surface of the hypotube to form the outer layer of the tool.

Also disclosed herein is a method of manufacturing a magnetic navigation enabled stylet configured for the delivery of an implantable medical lead. In one embodiment, the method includes: providing a hypotube; defining a recess in a wall of the hypotube, the recess extending longitudinally along the hypotube; positioning a sensor near a distal end of the hypotube; routing a conductor along the recess from the sensor towards a proximal end of the hypotube; and providing a fill material in the recess, the fill material imbedding at least part of the conductor in the recess.

Further disclosed herein is a method of manufacturing a magnetic navigation enabled stylet configured for the delivery of an implantable medical lead. In one embodiment, the method includes: providing a hypotube including a lumen and an outer circumferential surface; positioning a sensor near a distal end of the hypotube; routing a conductor along the outer circumferential surface from the sensor towards a proximal end of the hypotube; and extending a material over the conductor and outer circumferential surface of the hypotube and forming an outer layer of the stylet.

Also disclosed herein is a method of implanting a medical lead. In one embodiment, the method includes: providing a magnetic navigation enabled guidewire having a sensor near a distal end of the guidewire; providing a magnetic navigation enabled stylet having a sensor near a distal end of the stylet; positioning the guidewire distal end near a lead implantation site and sensing the location of the sensor of the guidewire; employing the stylet distal end to push the medical lead over the positioned guidewire towards the guidewire distal end; and sensing the location of the sensor of the stylet in relation to the sensor of the guidewire.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a side view of an embodiment of the tool.

FIG. 1B is a transverse cross section of the tool as taken along section line 1B-1B of FIG. 1A.

FIG. 2 is a side view of the patient in the magnetic field of the gMPS when the tool is located within the patient during the process of tracking a lead to the implantation site.

FIG. 3A is a side view of the hypotube employed as part of the tool of FIG. 1A.

FIG. 3B is a transverse cross section of the hypotube as taken along section line 3B-3B in FIG. 3A.

FIG. 4A is a side view of another embodiment of the tool, wherein the one or more conductors are routed through a recess defined in the inner layer of the tubular body.

FIG. 4B1 is a transverse cross section of the tubular body as taken along section line 4B-4B in FIG. 4A, wherein the recess is a slot defined completely through the wall of the inner layer.

FIG. 4B2 is a transverse cross section of the tubular body as taken along section line 4B-4B in FIG. 4A, wherein the recess is a groove defined in the outer circumferential surface of the inner layer.

FIG. 5A is a side view of a hypotube used in the embodiment of the tool depicted in FIG. 4A.

FIG. 5B1 is a transverse cross section of the hypotube used in the embodiment of the tool depicted in FIG. 4B1, wherein the cross section is taken along section line 5B-5B of FIG. 5A.

FIG. 5B2 is a transverse cross section of the hypotube used in the embodiment of the tool depicted in FIG. 4B2, wherein the cross section is taken along section line 5B-5B of FIG. 5A.

FIG. 6 is a longitudinal cross section of an embodiment of the tool, wherein a portion of the tubular body of the tool is formed from a plated metal.

FIG. 7 is a longitudinal cross section of an embodiment of the tool, wherein a portion of the tubular body of the tool is formed from a coil wound from flat wire.

FIG. 8 is a longitudinal cross section of an implantable medical lead being tracked over a guidewire via a stylet to a lead implantation site within the coronary venous anatomy of the patient, wherein the guidewire and stylet are both equipped with passive sensor coils that can be sensed with respect to both position and orientation within an active field of a gMPS.

### DETAILED DESCRIPTION

A magnetic navigation enabled (“MNE”) tubular delivery tool 10 is disclosed herein, along with its methods of manufacture and use. The tool 10 is configured for the delivery of an implantable medical lead 15 to a lead implantation site 20 within a patient 25. The tool 10 may be in the form of a stylet, catheter, sheath or other tubular body and is configured so as to be capable of being tracked within the patient 25 via a guided medical positioning system (“gMPS”) 30 such as the gMPS as manufactured by St. Jude Medical's MediGuide Ltd. of Haifa, Israel (see, e.g., U.S. Pat. No. 6,233,476 and U.S. patent application Ser. No. 10/458,332, which are incorporated by reference herein in their entireties). More specifically, due to tool's configuration and use with the gMPS 30, the tool 10 and the cannulation, lead delivery and lead placement made possible via the tool 10 can be tracked in real time.

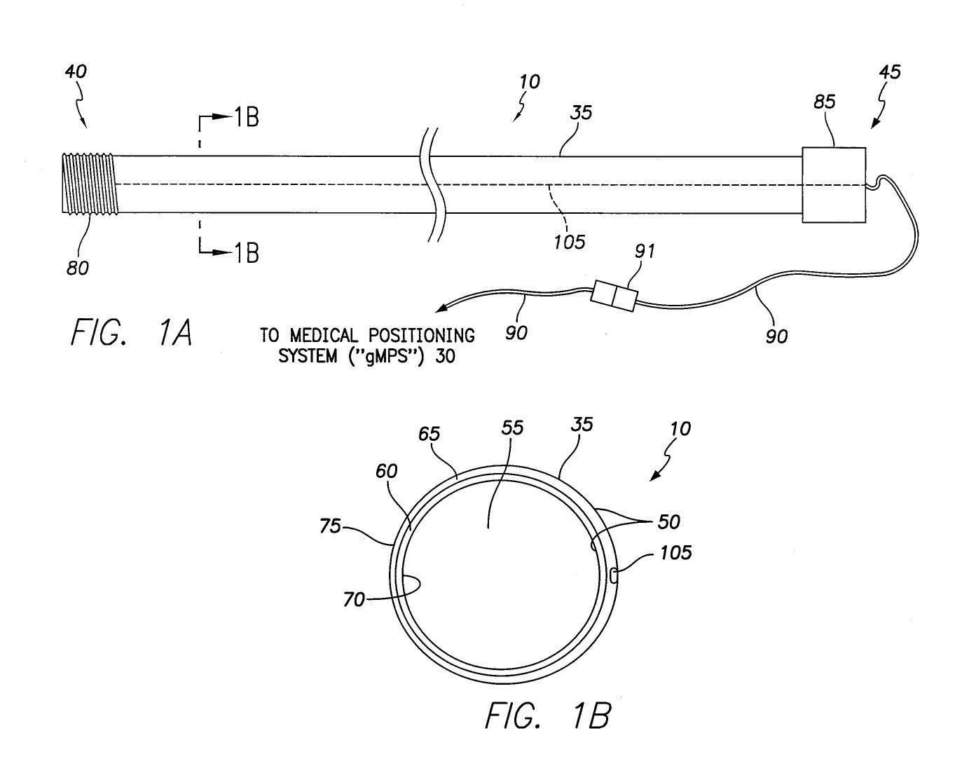

As depicted in FIGS. 1A and 1B, which are, respectively, a side view and a transverse cross section of the tool 10, in one embodiment, the tool 10 includes a tubular body 35 having a distal end 40, a proximal end 45, a wall structure 50, and a central lumen 55 extending between the proximal end 40 and distal end 45. The wall structure 50 includes an inner layer 60, an outer layer 65 extending about the outer circumferential surface of the inner layer 60, an inner circumferential surface 70 that defines the lumen 55, and an outer circumferential surface 75.

As shown in FIG. 1A, in one embodiment, the distal end 40 includes a passive sensor coil 80, which is supported on the tubular body 35. In one embodiment, the passive sensor coil 80 has four layers of 58 AWG coiling cable that is 2 mm long. In another embodiment, the passive sensor coil has four layers of 60 AWG cable that is 4 mm long. In some embodiments, the sensor will have a wire size range of between approximately 58 AWG and approximately 60 AWG, with an overall length range of between approximately 2 mm and approximately 6 mm, and a diameter range of up to approximately 0.018″. Magnetic wire may be employed for the coil while Mu metal is employed for the core or base material.

As illustrated in FIG. 1A, in one embodiment, the proximal end 45 includes a hub 85 that may be employed by the physician for the handling and manipulation of the tool 10. A cable 90 extends from the hub 85. The cable 90 may include a well shielded quick connect 91, which may be located at the hub 85 or anywhere along the length of the cable 90. The hub 85 may include a sensor port into which the cable 90 is received. The cable 90 may be shielded and be sufficiently long to extend outside the active magnetic field 95 of the gMPS 30 to reduce the introduction of electronic noise.

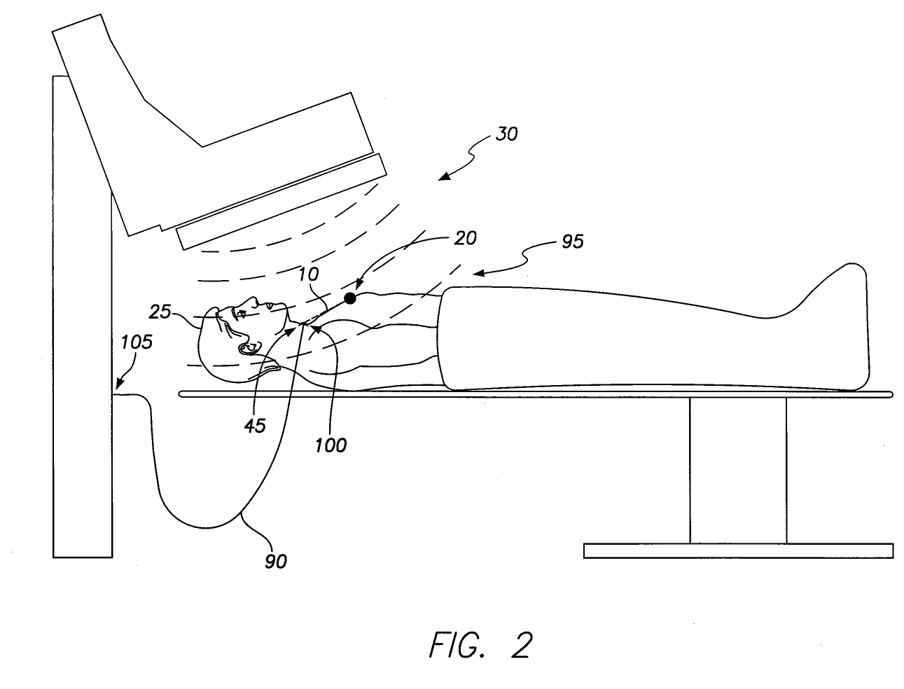

As indicated in FIG. 2, which is a side view of the patient 25 in the magnetic field 95 of the gMPS 30 when the tool 10 is located within the patient 25 during the process of tracking a lead to the implantation site 20, the tool proximal end 45 projects from an access site 100, and the cable 90 extends between the tool proximal end 45 and the gMPS cable connection location 105.

As indicated in FIGS. 1A and 1B, one or more conductors (e.g., jacketed or non-jacketed solid wire, jacketed or non-jacketed multi-filar cable, etc.) 105 extend through the wall structure 50 of the tubular body 35 from the sensor coil 80 to the coupling of one or more conductors 105 to the cable 90 at the hub 85. In one embodiment, as can be understood from FIG. 1B, the inner layer 60 is a polytetrafluoroethylene “PTFE” layer supported on a mandrel during assembly of the tubular body 35, the one or more conductors 105 extends along the inner layer 60, and the outer layer 65 is a polymeric jacket is deposited over the inner layer 60 and in which the one or more conductors 105 are imbedded. Once assembled, the entire tubular body 35 is then removed from the mandrel.

As illustrated in FIG. 1B, in one embodiment, the inner layer 60 is formed of a hypotube made of a non-magnetic metal material (e.g., platinum, gold, palladium, etc.) and the outer layer 65 is formed of a polymer material (e.g., polyimides, polyamides, PET, PTFE, pellethane, etc.). The one or more conductors 105 are imbedded in the outer layer 65. In other embodiments, to intensify magnetic signal, the hypotube is made of or includes a Mu metal (i.e., an alloy with high magnetic permeability) or stainless steel, and the outer layer is formed of a polymer.

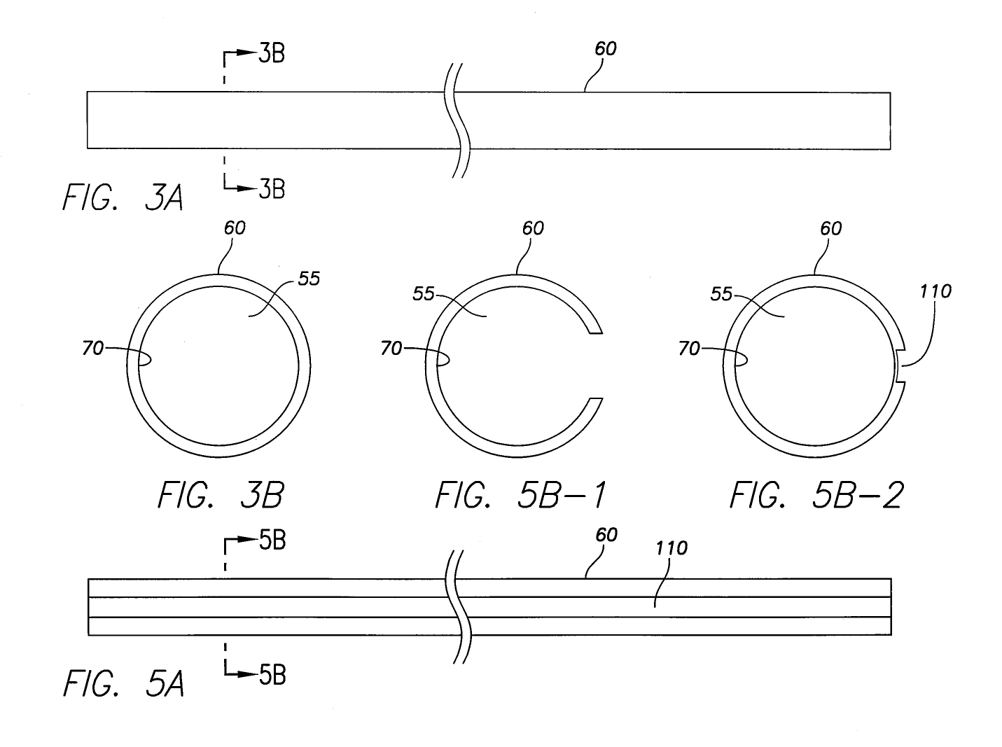

As can be understood from FIGS. 3A, which are respectively a side view of the hypotube 60 and a transverse cross section of the hypotube 60 as taken along section line 3B-3B in FIG. 3A, in one embodiment of the assembly process for the tool 10 of FIG. 1A, the hypotube 60 is provided without the outer layer 65. The sensor 80 is then mounted on the distal end of the hypotube 60 and the one or more conductors 105 are routed along the hypotube 60 from the sensor to the proximal end of the hypotube 60. The hub 45 is mounted on the proximal end of the hypotube 60, the proximal end of the one or more conductors 105 being coupled to a sensor port in the hub 45 or otherwise configured to allow the proximal end of the one or more conductors 105 to be coupled to the cable 90. The polymer layer 65 may then be pulled, reflowed, spray-deposited, or otherwise provided about the outer circumferential surface of the hypotube 60 to form the outer layer 65. In some embodiments, the outer layer 65 may be formed of a non-magnetic metal (e.g., platinum, gold, palladium, etc.) that is plated over the one or more conductors 105 and the inner layer 60. Where the outer layer 65 is sprayed, plated or otherwise deposited over the inner layer 60, the circumferential surface 75 may be subjected to a grinding process to achieve a uniform outer circumferential surface 75. The result of the aforementioned processes of providing the outer layer 65 about the inner layer 60 is the one or more conductors 105 being imbedded in the outer layer 65.

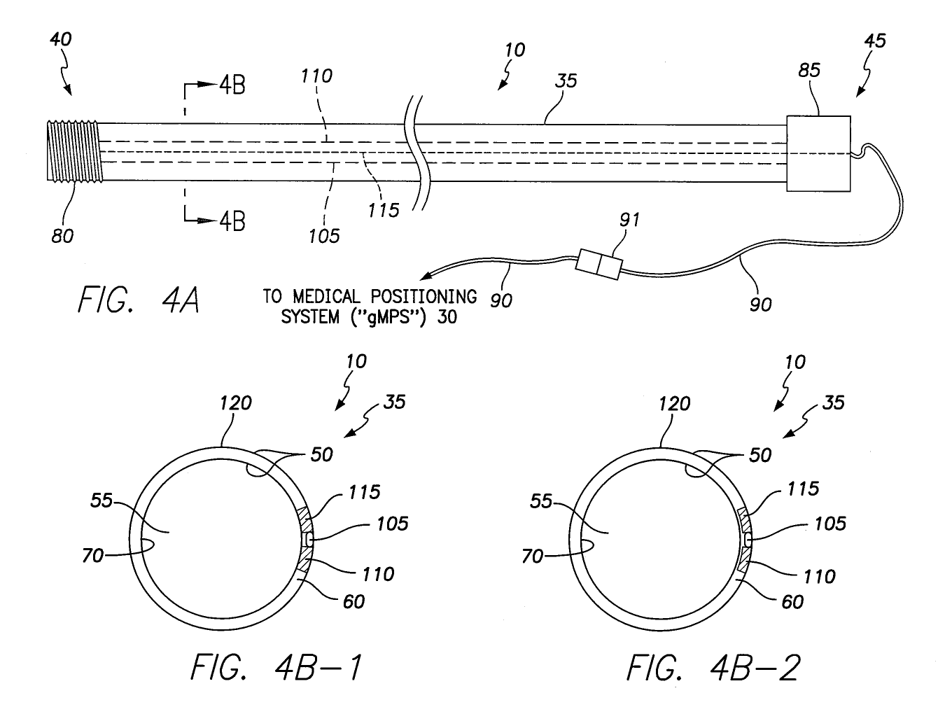

In some embodiments, the one or more conductors 105 are located at least partially within the wall thickness of the inner layer 60. For example, as depicted in FIG. 4A, which is a side view of another embodiment of the tool 10, a longitudinally extending recess 110 may be defined in the hypotube 60 between the distal end 40 and the proximal end 45 of the tool 10. In some embodiments, there may be two or more such recesses 110 defined in the hypotube 60. The one or more conductors 105 are routed through the recess 110 or recesses 110. Specifically, as depicted in FIG. 4B1, which is a transverse cross section of the tubular body 35 as taken along section line 4B-4B in FIG. 4A, in one embodiment, the recess 110 is a slot 110 extending completely through the wall of the hypotube 60. In other words, as depicted in FIGS. 5A and 5B1, which are, respectively, a side view and a transverse cross section of the hypotube 60 used in the embodiment of the tool 10 depicted in FIGS. 4A and 4B1, the recess 110 is a slot 110 extending completely through the wall of the hypotube 60, resulting in hypotube 60 having a C-shaped cross section.

As can be understood from FIG. 4B1, the one or more conductors 105 are routed through the slot 110, and a polymeric coating or fill 115 is used to seal the one or more conductors 105 in the slot 110 and fill the slot 110 in such a manner that the outer circumferential surface 120 of the hypotube 60 is generally uniform and free of voids. The outer circumferential surface 120 of the filled hypotube 60 may then be subjected to a grinding process to make the outer circumferential surface 120 uniform. Alternatively or additionally, an outer layer 65 similar to that of FIG. 1B may be provided about the outer circumferential surface 120 of the hypotube 60. The polymeric coating or fill 115 depicted in FIG. 4B1 may be a polymer material such as, for example an ultraviolet (“UV”) cured polymeric material, PELLETHANE® or Dymax 203, 207, or 1128. The resulting embodiment depicted in FIG. 4B1 is a tool 10 having a tubular body 35 with the one or more conductors 105 imbedded in the wall structure 50 of the tubular body 35 and, more specifically, at least partially imbedded in the wall structure of the hypotube 60.

In another embodiment, as depicted in FIG. 4B2, which is a transverse cross section of the tubular body as taken along section line 4B-4B in FIG. 4A, the recess 110 is a groove 110 defined in the outer circumferential surface 120 of the hypotube 60. In other words, as depicted in FIGS. 5A and 5B2, which are, respectively, a side view and a transverse cross section of the hypotube 60 used in the embodiment of the tool 10 depicted in FIGS. 4A and 4B2, the recess 110 is a groove 110 in the outer circumferential surface 120 that does not extend completely through the wall of the hypotube 60, resulting in the hypotube 60 having a wall portion with a notched cross section.

As can be understood from FIG. 4B2, the one or more conductors 105 are routed through the groove 110, and a polymeric coating or fill 115 is used to seal the one or more conductors 105 in the groove 110 and fill the groove 110 in such a manner that the outer circumferential surface 120 of the hypotube 60 is generally uniform and free of voids. The outer circumferential surface 120 of the filled hypotube 60 may then be subjected to a grinding process to make the outer circumferential surface 120 uniform. Additionally or alternatively, an outer layer 65 similar to that of FIG. 1B may be provided about the outer circumferential surface 120 of the hypotube 60. The polymeric coating or fill 115 depicted in FIG. 4B2 may be a polymer material such as, for example UV, Pellethane, etc. The resulting embodiment depicted in FIG. 4B2 is a tool 10 having a tubular body 35 with the one or more conductors 105 imbedded in the wall structure 50 of the tubular body 35 and, more specifically, at least partially imbedded in the wall structure of the hypotube 60.

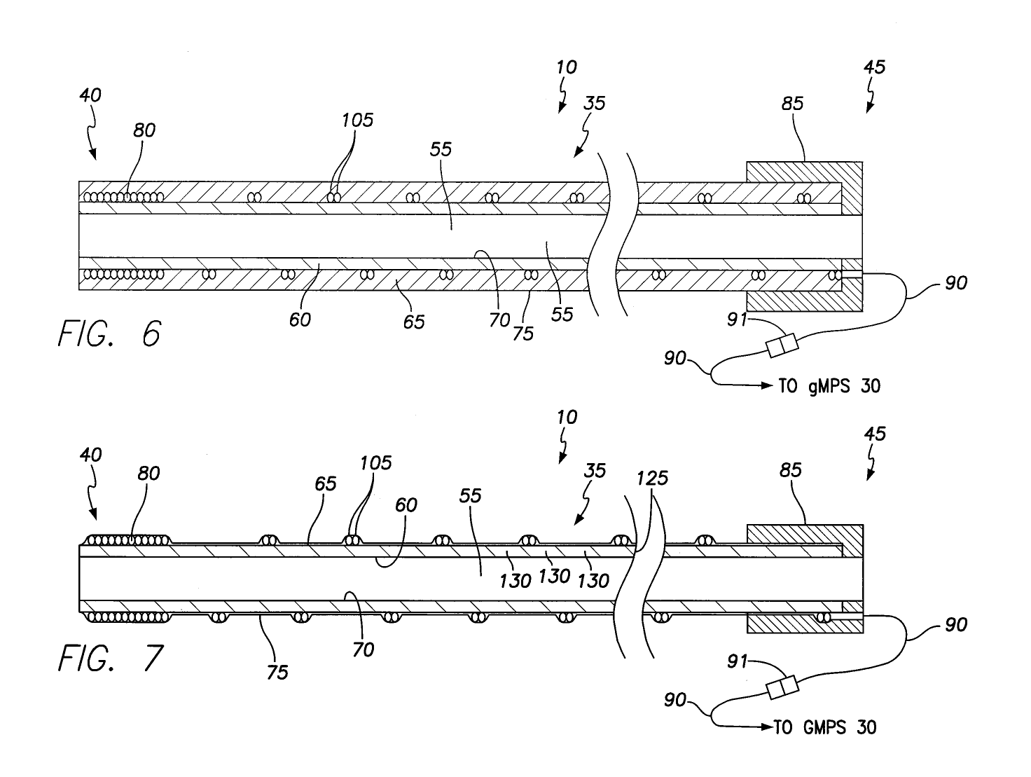

While the embodiment depicted in FIGS. 1A and 4A illustrate the one or more conductors 105 being routed along the hypotube 60 in a generally direct, straight route, in other embodiments, the one or more conductors 105 may have other routing configurations along the hypotube 60. For example, as depicted in FIG. 6, which is a longitudinal cross section of an embodiment of the tool 10, the conductors 105 are helically routed along the inner layer 60 of the tubular body 35 of the tool 10. The inner layer 60 is formed of a thin walled material, such as, for example, polyimide tube having a wall thickness of approximately 0.00025″. The sensor coil 80 is wound over the distal end of the inner layer 60, and the conductors 105 are connected to the coil 80, for example, via soldering. The conductors 105 are helically routed along the length of the inner layer 60 from the sensor coil 80 on the distal end 40 of the inner layer 60 to the connection with the cable 90 at the hub 85 on the tubular body proximal end 45.

As indicated in FIG. 6, an outer layer 65 of metal is plated over the outer circumferential surface of the inner layer 60 and the coil 80 and conductors 105 located thereon. In one embodiment, the plating process may include several steps, including applying a sputtering coat of base metal over the assembly of the inner layer 60, coil 80 and conductors 105, thereby forming the outer layer 60. In one embodiment, the plating process coats the components (i.e., the coil 80 and conductors 105) on the inner layer 60 with a uniform thickness of metal. Thus, the portions of the outer layer 65 (i.e., metal plating layer) extending over the coil 80 and conductors 105 have a diameter that exceeds the diameter of the portions of the outer layer 65 that simply extends directly over the outer circumferential surface of the inner layer 60 (i.e., does not extend over the coil 80 and conductors 105). A grinding process can then be employed to cause the resulting tubular body 35 and, more specifically, the outer circumferential surface 75 of the resulting tubular body 35 to have a uniform outer diameter. The resulting tubular body 35 will be a composite wall structure having an outer metal surface, the sensor 80 and conductors 105 buried in the wall structure, an inner circumferential surface 70 with an uniform inner diameter, and an outer circumferential surface 75 with an uniform outer diameter. In some embodiments, the tubular body 35 may have a wall thickness of approximately 0.002″.

In some embodiments, the metal coating 65 may be electrically coupled to a ground wire. Accordingly, the metal coating 65 will not adversely impact the operation of the sensor 80, but may provide some shielding against unwanted electrical noise.

In one embodiment, a tool 10 as described above with respect to FIG. 6 may be sized and configured for use as a stylet 10. In other embodiments, the tool 10 as described above with respect to FIG. 6 may be sized and configured for use as a catheter, sheath or other tubular tool for the delivery of implantable medial leads.

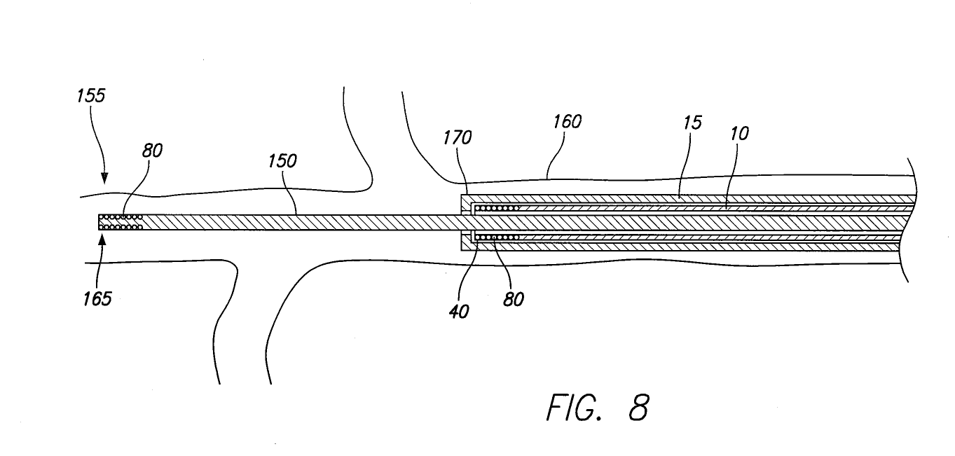

Another embodiment of the tool 10 also employs helically routed conductors 105, as discussed below with respect to FIG. 7, which is a longitudinal cross section of such an embodiment of the tool. As shown in FIG. 7, the inner layer 60 of the tool tubular body 35 is formed of a flat wire 125 wound into a coil, wherein the adjacent coils 130 of the flat wire 125 generally abut each other to form a cylindrical tubular inner layer 60. The inner circumferential surface 70 of the inner layer 60 defines a lumen 55. The conductors 105 are helically routed along the length of the inner layer 60 from the sensor coil 80 on the distal end 40 of the inner layer 60 to the connection with the cable 90 at the hub 85 on the tubular body proximal end 45. An outer layer 65 formed of a heat shrink material is provided about the conductors 105, sensor coil 80, and inner layer 60. The heat shrink material 65 may be a thin wall (approximately 0.00025″) Polyethylene Terephthalate (“PET”) and may be nearly inelastic. Once heat shrunk about the inner layer 60, helically wound conductors 105 and the sensor coil 80, the outer layer 65 formed of the heat shrink material causes the entire tubular body assembly 35 to be generally rigid, much like a metal tubular body. The outer layer 65 forms the outer circumferential surface 75 of the tubular body 35 of the tool 10.

In one embodiment, the flat wire 125 has a cross section that is approximately 0.003″ by approximately 0.0007″ and forms an inner layer 60 with a wall thickness of approximately 0.0007″. The sensor coil 80 wound on the distal end of the tubular body 35 may be formed of approximately 60 gauge (approximately 0.0004″ diameter) copper wire. The conductors 105 may be approximately 54 gauge (approximately 0.001″ diameter) cable connected (e.g., via soldering) to the wire of the sensor coil 80 and helically wound about the inner layer 60 from the sensor 80 to the hub 85 and the connection with the cable 90.

In one embodiment, a tool 10 as described above with respect to FIG. 7 may be sized and configured for use as a stylet 10. In other embodiments, the tool 10 as described above with respect to FIG. 7 may be sized and configured for use as a catheter, sheath or other tubular tool for the delivery of implantable medial leads.

In one embodiment, as can be understood from FIGS. 1A and 2, the gMPS 30 as manufactured by MediGuide generates a magnetic filed 95 that can accurately sense the position and orientation of a passive sensor coil 80 within the active field 95. The gMPS 30 incorporates the real time magnetically sensed orientation and position (“O&P”) of the sensor coil 80 and registers the O&P onto previously recorded fluoroscopic images/cines. As a result, the physician can utilize one time recordings of fluoroscopic images/cines in conjunction with real time projected P&O of the sensor coil 80 to track the progress of the tool 10 within the patient 25.

In some embodiments, the gMPS 30 employs two different venogram images with an angle of separation of greater than 45 degrees to be used to generate a three dimensional (“3D) representation of the geometry of the patient's vasculature and cardiac structure. The P&O of the tool 10 can be projected onto the 3D representation of the patient's coronary venous anatomy, thereby providing the physician a better understanding of the P&O of the tool 10 within the patient's coronary venous anatomy.

In one embodiment, as can be understood from FIG. 8, which is a longitudinal cross section of an implantable medical lead 15 being tracked over a guidewire 150 via a stylet 10 to a lead implantation site 155 within the coronary venous anatomy 160 of the patient 25, both the guidewire 150 and stylet 10 are equipped with passive sensor coils 80 at their respective distal ends 40, 165. The guidewire 150 is navigated through the patient's coronary venous anatomy 160 until the guidwire distal end 165 is positioned as desired at the lead implantation site 155. As the guidewire distal end 165 includes a sensor coil 80, the gMPS is able to sense both P&O of the guidewire distal end 165 as it is navigated through the patient's coronary venous anatomy 160.

As can be understood from FIG. 8, the lead 15 is tracked over the positioned guidewire 150, the lead 15 coaxially extending over the guidewire 150. To cause the lead 15 to distally displace over the guidewire 150, the stylet 10 may be coaxially extended over the guidewire 150 and coaxially enclosed within the lead 15, the distal end 40 of the stylet 10 abutting the interior of the distal end 170 of the lead 15. Thus, distal force exerted on the stylet proximal end 45 by the physician can be transferred to the lead distal end 170 by the stylet distal end 40 abutting against the interior of the lead distal end 170, thereby causing the lead 15 to distally travel along the guidewire 150 to the lead implantation site 155. As the stylet distal end 40 includes a sensor coil 80, the gMPS is able to sense both P&O of the stylet distal end 40 as it and the lead distal end 170 travel along the guidewire 150. Since the stylet distal end 40 and its sensor coil 80 are in close proximity to the lead distal end 170 as the stylet 10 is used to push the lead 15 along the guidewire 150, the physician, via the gMPS 30 is provided with an good understanding of the location of the lead distal end 170 relative to the lead implantation site 155, thereby facilitating the implantation of the lead 15.

Although the present invention has been described with reference to preferred embodiments, persons skilled in the art will recognize that changes may be made in form and detail without departing from the spirit and scope of the invention.

What is claimed is:

1. A magnetic navigation enabled tool configured for the delivery of an implantable medical lead, the tool comprising:
a hypotube including a recess defined in a wall of the hypotube and extending longitudinally along the hypotube;
a sensor near a distal end of the hypotube;
a conductor routed along the recess from the sensor towards a proximal end of the hypotube; and
a fill material imbedding the conductor in the recess and generally filling the recess.

2. The tool of claim 1, wherein the recess includes a groove that extends only partially through the wall of the hypotube from an outer circumferential surface of the hypotube.

3. The tool of claim 1, wherein the recess includes a slot that extends completely through the wall of the hypotube from an outer circumferential surface of the hypotube to an inner circumferential surface of the hypotube.

4. The tool of claim 1, wherein the fill material is generally limited in location to the recess.

5. The tool of claim 1, wherein the fill material is part of a material forming a layer extending about an outer circumferential surface of the hypotube.

6. The tool of claim 1, wherein the sensor is passive and includes a coil.

7. The tool of claim 1, wherein the tool is a stylet.

8. A magnetic navigation enabled tool configured for the delivery of an implantable medical lead, the tool comprising:
a hypotube including a lumen and an outer circumferential surface;
a sensor near a distal end of the hypotube;
a conductor routed along the outer circumferential surface from the sensor towards a proximal end of the hypotube; and
a material extending over the conductor and outer circumferential surface of the hypotube and forming an outer layer of the tool.

9. The tool of claim 8, wherein the material is a thin wall heat shrink material.

10. The tool of claim 9, wherein the hypotube is at least partially formed of a helically wound flat wire, the heat shrink material at least partially contributing to the helically wound flat wire being held in the form of a cylindrical hypotube.

11. The tool of claim 8, wherein the material is at least one of reflowed, extruded or sprayed about the outer circumferential surface of the hypotube, the conductor being imbedded in the material.

12. The tool of claim 8, wherein the material is a metal layer plated about the outer circumferential surface and the conductor.

13. The tool of claim 12, wherein an outer circumferential surface of the metal layer is the result of a grinding process.

14. The tool of claim 8, wherein conductor is helically routed along the outer circumferential surface.

15. The tool of claim 8, wherein the sensor is passive and includes a coil.

16. The tool of claim 8, wherein the tool is a stylet.

17. A method of manufacturing a magnetic navigation enabled stylet configured for the delivery of an implantable medical lead, the method comprising:
providing a hypotube;
defining a recess in a wall of the hypotube, the recess extending longitudinally along the hypotube;
positioning a sensor near a distal end of the hypotube;
routing a conductor along the recess from the sensor towards a proximal end of the hypotube; and
providing a fill material in the recess, the fill material imbedding at least part of the conductor in the recess.

18. The method of claim 17, wherein defining the recess in the wall of the hypotube includes creating a slot that extends completely through the wall of the hypotube from an outer circumferential surface of the hypotube to an inner circumferential surface of the hypotube.

19. The method of claim 17, wherein the fill material is generally limited in location to the recess.

20. The method of claim 17, wherein the fill material is part of a material forming a layer extending about an outer circumferential surface of the hypotube.

21. The method of claim 17, wherein the defining the recess in the wall of the hypotube includes creating a groove that extends only partially through the wall of the hypotube from an outer circumferential surface of the hypotube.

22. A method of manufacturing a magnetic navigation enabled stylet configured for the delivery of an implantable medical lead, the method comprising:
providing a hypotube including a lumen and an outer circumferential surface;
positioning a sensor near a distal end of the hypotube;
routing a conductor along the outer circumferential surface from the sensor towards a proximal end of the hypotube; and
extending a material over the conductor and outer circumferential surface of the hypotube and forming an outer layer of the stylet.

23. The method of claim 22, wherein the material is a thin wall heat shrink material.

24. The method of claim 23, further comprising forming the hypotube from a helically wound flat wire, wherein the extending the heat shrink material over the conductor and outer circumferential surface of the hypotube and forming the outer layer of the stylet at least partially contributes to the helically wound flat wire being held in the form of a cylindrical hypotube.

25. The method of claim 22, wherein extending the material is accomplished via at least one of reflow, extrusion or spraying about the outer circumferential surface of the hypotube, the conductor being imbedded in the material.

26. The method of claim 22, wherein extending the material is accomplished via plating a metal layer about the outer circumferential surface and the conductor.

27. The method of claim 26, further comprising grinding the outer surface of the metal layer.

28. The method of claim 22, wherein routing the conductor is done in a helical manner along the outer circumferential surface.

29. A method of implanting a medical lead, the method comprising:
providing a magnetic navigation enabled guidewire having a sensor near a distal end of the guidewire;
providing a mangnetic navigation enabled stylet having a sensor near a distal end of the stylet;
positioning the guidewire distal end near a lead implantation site and sensing the location of the sensor of the guidewire;
employing the stylet distal end to push the medical lead over the positioned guidewire towards the guidewire distal end; and
sensing the location of the sensor of the stylet in relation to the sensor of the guidewire.
